(19) United States
(12) Patent Application Publication
(10) Pub. No.: US 2015/0196677 A1
(43) Pub. Date: Jul. 16, 2015

(54) Air Freshener Composition and Methods Thereof

(71) Applicant: Agilex Flavors & Fragrances, Inc., Piscataway, NJ (US)

(72) Inventors: Florin-Iosif Vlad, Annandale, NJ (US); Frank Pietoso, Hamilton Square, NJ (US); Heather Elisa Adams, Raritan, NJ (US); Natalie Lois Hinden-Kuhles, Somerville, NJ (US)

(21) Appl. No.: 14/593,276

(22) Filed: Jan. 9, 2015

Related U.S. Application Data
(60) Provisional application No. 61/925,897, filed on Jan. 10, 2014.

(51) Int. Cl. A61L 9/01; A61L 9/03

(52) U.S. Cl. CPC A61L 9/01; A61L 9/03

(57) ABSTRACT

Disclosed in certain embodiments is an air freshener composition comprising a matrix comprising a fragrance dispersed in a thermo-reversible cross-linked polymer, the matrix having a melting point of at least 120° F.

The present application claims priority to U.S. Provisional Patent Application No. 61/925,897 filed Jan. 10, 2014, the disclosure of which is hereby incorporated by reference.

### BACKGROUND OF THE INVENTION

From a consumer standpoint, a need has existed, and continues to exist, for a safe environment that also offers a persistently pleasant ambiance due to aroma or fragrance presence. It has therefore been a challenge and objective for air-freshener manufacturers to provide long-lasting aroma of fragrance releasing products that bring comfort and wellness.

Air-fresheners are common devices used to improve and/or to change the olfactory characteristics of an environment through a continuous, also known as sustained, or controlled fragrance release mechanism. The difficulty in achieving these objectives lies in fragrance complexity and subjective sensorial perceptions of fragrance. Fragrances are created with ingredients that have different physical, chemical and organoleptic properties, such as volatilities, stability to external environmental conditions such as light or temperature, and threshold perception, respectively. In order for fragrances to be perceived, they must be volatilized and dispersed into the environment. Upon opening or activation of an air-freshener device, fragrance ingredients are released into the external environment mainly based on their volatility. The more volatile fragrance ingredients are released first, while the less volatile ones are delay released. Therefore, regardless of other specific molecular properties of the fragrance ingredients, the fragrance perception performance, such as strength and character, varies over time. By employing well selected fragrance carrying molecules, such as solvents with different solvency and controlling evaporation properties, and by entrapping the fragrance into a well-designed delivery system, such as a gel, the herein mentioned fragrance performing attributes can be enhanced over the life time of product using.

It has been recognized that it would be advantageous to develop an air-freshener that is esthetically pleasant, and hedonically performing for a desired length of time, such as 30 days, and that can have a more constant fragrance release. In addition, it has been recognized that is would be advantageous to develop an air-freshener that is safe in use without fear for spillage or self-ignition. It has been recognized it would be advantageous to develop a versatile air-freshener that allows the fragrance to be released through thermo-activation, air-forced circulation or a combination of those mechanisms. It has been also recognized it would be advantageous to develop an air-freshener that is portable and can be easily located or positioned.

### OBJECTS AND SUMMARY

It is an object of the present invention to provide a controlled release air freshener composition.

It is an object of certain embodiments of the present invention to provide a controlled release air freshener composition that is non-flowable.

It is a further object of certain embodiments of the present invention to provide a device comprising a controlled release air freshener composition in association with heat (e.g., with a heater or warmer).

It is another object of certain embodiments of the present invention to provide a device comprising a controlled release air freshener composition in association with a turbulence device.

It is a further object of certain embodiments of the invention to provide a method of manufacturing an air freshener composition or device as disclosed herein.

It is another object of certain embodiments of the present invention to provide a method of freshening the air comprising utilizing a composition or device as disclosed herein.

The above objects and others, may be met by the present invention which in certain embodiments is directed to an air freshener composition comprising a matrix comprising a fragrance dispersed in a thermo-reversible cross-linked polymer, the matrix having a melting point, e.g., of at least about 120° F.

In other embodiments, the invention is directed to an air freshener device comprising a composition as disclosed herein associated with a heat source.

In alternative embodiments, the invention is directed to a device comprising as disclosed herein associated with air circulating apparatus.

Certain embodiments of the invention are directed to a method of freshening air comprising heating a composition as disclosed herein such that fragrance is released into the environment.

Other embodiments are directed to a method of freshening air comprising generating turbulence onto a composition as disclosed herein such that fragrance is released into the environment.

For simplicity, in the present invention, the words aroma, fragrance, scent or perfume have similar meaning and are used indiscriminately.

### DETAILED DESCRIPTION

The present invention discloses a long-lasting, sustained or controlled release air-freshener composition in the form of a matrix comprising a fragrance and a polymer, e.g., a thermo-reversible cross-linked polymer. In a particular embodiment, the composition is in the form of a gel. In certain embodiments, the composition can be in association with heat and/or a turbulence apparatus (e.g., a fan) to facilitate the release of fragrance from the composition. The heater (warmer) and the turbulence apparatus can be electrically powered or be cordless.

In certain embodiments, the thermo-reversible polymer can incorporate high levels of fragrance, e.g., at least about 10%, at least about 20%, at least about 30%, at least about 40% at least about 50% or at least about 60% w/w, of the entire product composition.

In certain embodiments, the polymer content is less than about 50%, less than about 40%, less than about 30%, or less than about 20% w/w.

When a heating device, such as a heating plate is employed, the composition (e.g., in the form of a gel) softens under temperature continually release the fragrance for at least 5 days, at least 7 days, at least 21 days or at least 30 days. The highest limit of heating temperature is dictated by the softening point of the composition. During the life time of the product the gel of this invention will not turn it into a liquid flowing phase when used according to normal conditions. The herein mentioned air freshener is a non-spilling product compared with the similar melting wax air-freshener product, which will make the air-freshener gel product of this invention safer in application. When the fragranced gel of this invention is associated with a fragrance release device such as an air-flow vent, an air-flow ceiling fan, a fan, a heating product, or a device that contains both a heating element and a fan, which will enhance the fragrance release into the external environment, similar or better fragrance performance is achieved at equivalent level of fragrance compared to melting wax cubes. In the content of this disclosure, the fragrance performance is defined, e.g., as fragrance strength and character over a specified period of time, which is known as fragrance longevity, or long-lasting fragrance performance or fragrance integrity or linear fragrance release.

The turbulence device can be either an electrically powered product or a cordless, portable battery operated product. In certain embodiments, air-freshener device may be have the capability to control the fragrance release through specific product design and by incorporating mechanical controlling elements, such as switching knobs for the heat source or fan. The external environment might be a room, a locker, or any external space designated for permanent or temporal living such as a tent, cabin or RV.

In certain embodiments, the invention provides an air-freshener delivery system with a scent entrapped within a translucent to clear, resilient, thermo-reversible cross-linked polymeric matrix gel. The scent is capable of dispersing out of the gel to surrounding environment through thermo-diffusion and/or air-forced circulation mechanisms.

The fragrance gel compositions of the present invention confer unexpected long-lasting fragrance performance in both passive and dynamic delivering devices. Active devices can be any warmer, either electrically or flame powered. It may also be a portable cordless device, with a heating plate or any surface incorporated or attached to it that can emanate heat to the said fragranced gel. Active devices can also be any device capable to force or circulate air over or around the said fragrance gel through different mechanisms such as air convection generated by a fan.

In certain embodiments, the present disclosure is directed to a clear, transparent or translucent polymeric thermo-reversible fragrance gel comprising up to 50% w/w fragrance, or other active ingredients such as malodor counteracting, malodor neutralizing or masking agents, electrostatic neutralizing ingredients, mold controlling or mold neutralizing ingredients, or mixtures of fragrances with such kinds of active agents. The fragrance gel may comprise from about 0.1 to about 30% w/w normal, branched, cross-linked or radial diblock, triblock, or multiblock copolymers based on thermoplastic rubbers or mixtures. A fragrance carrier, such as a solvent or blend of solvents with high fragrance solvency and evaporation controlled characteristics may be employed to achieve the fragrance performance, which can be defined as fragrance longevity in strength and character. The fragrance carrier may be, e.g., a paraffin oil, straight chain hydrocarbon molecules, isoparaffic oil, mineral oil, or blends of thereof. Optionally, the composition may comprise one or more polymer compatible suspending or dispersing agents for solids and/or liquids.

In certain embodiments, the fragrance composition of the present invention is a thermo-reversible cross-linked polymeric matrix with a high melting point, e.g., higher than about 120° F. or higher than about 150° F. or higher than about 170° F., in order to soften under heating, while releasing the fragrance, but to remain solid and keep its clarity or transparency. The composition can release fragrance in a persistent or controlled way over extended period of times. When using a heating source device, such as a heating plate or a device that can force air circulation, such as a fan, the gel of this invention can release fragrance, e.g., up to a month, as compared with the 72 hours performance of fragranced melting wax cubes.

In certain embodiments, the invention is directed to a translucent to clear thermo-reversible fragrance gelled into a mixture of evaporation and firmness controlling agents such as polymers, fragrance carrier solvents and optionally dispersing solvents. The composition may comprise in certain embodiments:
(i) From 0.1 to 90% w/w fragrance, optionally containing one or more fragrance miscible or fragrance dispersible actives such as malodor counteracting, malodor neutralizing or malodor masking agents, dyes, oil-soluble pigments, phosphorescent and/or fluorescent additives, glitter, pearl, insect repelling compounds, antifungal ingredients, antibacterial actives, antimicrobial actives, UV and light stabilizer ingredients or mixtures thereof;
(ii) From about 0.1 to 50% w/w normal, branched, cross-linked or radial diblock, triblock, or multiblock copolymers based on thermoplastic rubbers or blends thereof comprising from about 0 to 100% w/w of one or more diblock copolymer and from about 100 to 0% w/w of one or more triblock copolymer, branched, cross-linked, radial and/or multiblock polymer;
(iii) From about 0.1 to 75% w/w fragrance carrier such as a paraffin oil, straight chain hydrocarbon molecules, isoparaffic oil, mineral oil, or blends of thereof; and
(iv) Optionally, whenever is necessary less than 20% w/w polymer compatible aliphatic and aromatic suspending or dispersing agent for fragrance insoluble or fragrance-immiscible solids and/or liquids or mixtures thereof;

In one embodiment, the copolymers or blends thereof comprise Hydrogenated Styrene-Isoprene-Butadiene copolymers and/or Styrene-Ethylene/Butylene-Styrene, or Styrene-Ethylene/Propylene-Styrene copolymers.

The compositions of the invitation, e.g., gels, are generally non-aqueous. However, water may be optionally added to certain compositions to impact the fragrance performance.

Commercial available thermoplastic elastomers that may be used in the present invention are sold under different trade names, such as Kraton® by Shell Chemical Company, Styroflex® by BASF, Calprene® by Dynasol, or SEPTON® by Kurary.

The thermoplastic Kraton® elastomers display unique combination of physical and mechanical properties such as high strength and low viscosity due to their specific molecular structures of linear diblock, triblock and radial copolymers. Each molecule of Kraton® elastomer consists of block segments of styrene units and rubber monomer and/or co-monomer units such as isoprene, ethylene or butadiene. Each of the said blocks may consist of at least 100 monomer or co-monomer units. The most common structures are Styrene-Butadiene-Styrene (SBS) or Styrene-Isoprene-Styrene (SIS) elastomers. However, of special interest for this invitation is a Kraton® AG elastomer, in particular Kraton® AG-51 elastomer, which has polystyrene end blocks and elastomeric midblocks. The Kraton® AG elastomer is the type ABA triblock copolymer. The styrene blocks offer mechanical resistance to the gel through cross-linked domain, while the rubber polybutadiene midblocks elasticity and oil solubility.

Another triblock elastomer that may be employed in the formation of the invention is SEPTON®, which is a type ABC triblock copolymer, which consists of hydrogenated styrene-isoprene-butadiene copolymer. The preferred ABC triblock copolymer is SEPTON® 4055.

When fragrance is added to the polymeric matrix, a fragrance carrier comprising a solvent or blend of solvents with high fragrance solvency and evaporation controlled characteristics may be employed from about 0.1 to 75% w/w of the total composition.

While not being limited by theory, the herein mentioned fragrance carrier solvents are selected based on their fragrance solvency and evaporation controlled characteristics. The fragrance carrier solvents useful in the compositions of the invention, include without limitation, paraffin oils, mineral oils, isoparaffinic oils, animal oils, vegetable oils and mixtures thereof.

Commercial available fragrance carrier solvents that may be utilized in the present invention are sold under different trade names, such as Isopar® by ExxonMobil Chemical, Drakeol® by Penreco, Soltrol® by Chevron-Phillips, Linpar® by Sasol, or FlexiSolve™ by INVISTA.

One fragrance carrier employed in the invention is isoparaffinic oils or blends thereof. One bend may be Isopar M and Isopar L in different weight ratios. In one embodiment, the fragrance carrier is saturated paraffin mixtures in the C₁₄-C₁₆ carbon range provided by Sasol under Linpar® trade names. They are available in both hydrogenated (LINPAR® 1416V) and un-hydrogenated (C₁₄₋₁₆ paraffins) versions.

The fragranced compositions of the invention may contain one or more fragrance materials such as natural and/or synthetic fragrance raw materials. Of particular interest are oil soluble perfume oils, which may or may not be in mixture with water soluble perfume oils. The oil soluble perfume oils are natural, or natural-identical essential oils such as orange oil, lavender oil, pine oil, eucalyptus oil, lemon oil, clove leaf, peppermint oil, cedarwood oil, rosemary oil, bergamot oil, lavandin oil, patchouli oil, chamomile oil, jasmine oil, spike oil, rose oil, Vetiver oil, fennel oil, anise oil, thyme oil, germanium oil, menthol, and marjoram oil. An animal fragrant is for example musk, castoreum, aber or zibet. Spagyric essences are also known in the art. They are made by fermenting certain herbs that are then processed to the final product. Synthetic fragrance ingredients are for example synthetic essential oils such as composed of single compounds such as linalol, terpineol, nerol, citronellal, benzaldehyde, cinnamon aldehyde, vanillin, ethylvanillin, or methylacetophenone. The fragrance materials may also be synthetic oil-soluble perfume oils selected from the usual group consisting of fragrant hydrocarbons, alcohols, ketones, aldehydes, ethers, esters, polyene derivatives. Naturally, this term also encompasses any mixture of perfume oils previously described, or perfume concentrates. Diluents well-known to those of skill in the art may be used in combination with fragrance ingredients and/or essential oils. As used in the present invention, the term “fragrance”, “fragrance composition” or “perfume” refers to a blend of individual perfumery materials (ingredients). The individual perfumery materials and accords suitable for use in the gels of the present invention are catalogued and described in references and databases well-known to those of skill in the art including the following: S. Arctander, Perfume and Flavor Chemicals, Volumes I and II (1960, 1969; reprint 2000); Allured's Flavor and Fragrance Materials (2005); and database maintained by the Research Institute for Fragrance Materials at www.rifm.org.

According to certain embodiments of the invention, one or more hydrocarbon-soluble substances, or mixtures thereof, may be optionally added to act as suspending agents or dispersant for solids or liquids. The suspending or dispersible solvents may be, but not limited to mineral oils, petrolatum, synthetic hydrocarbons and silicon oils. The solids or liquids to be suspended or dispersed in the composition comprise any non-hydrocarbon oil liquid which will disperse into the gel and remain substantially suspended or even dispersed therein. Examples of solids can be suspended in the compositions of this invention comprise organic and/or inorganic pigments, phosphorescence materials, fluorescence materials dyes, and capsules containing different ingredients,

Examples of oil-immiscible liquids which can be dispersed in the gel of this invention comprise water, glycol solvents, such as propylene glycol or dipropylene glycol, surfactants, emulsifiers, polar esters, fluorinated compounds and mixtures thereof.

In certain embodiments, the gel consistency of the invention is controlled by varying the amount, ratio and types of certain polymers, such as triblock copolymers with different compositions and distributions, and the level of fragrance and optional fragrance carrier solvent.

In certain embodiments, the fragranced compositions of the invention are a result of physical-crosslinking of the rigid segments of the triblock copolymers that form sub-microscopic domains, which are distributed into a three-dimensional network, and are capable to entrapping fragrance ingredients into the interspaces created through the lattice structure. Crosslinking of the insoluble domains can be controlled by factors affecting the crosslinking density of the resulting network including length of insoluble block domains, length of the soluble block domains, and the number of crosslinking sites. For example, branched or star copolymers have more crosslinking sites and because of this they will form more rigid stiff gels than linear or less branched polymers. The fragrance, fragrance carrier solvent, and dispersing solvent, when present, will act as plasticizers. The plasticizing effect strongly depends on the composition and level of fragrance, fragrance carrier and dispersing solvent, when present.

Certain fragranced compositions exhibit syneresis, wherein the separation of fragrance from the gel occurs. The synerisis depends on the polymer type and amount, fragrance type and amount, fragrance carrier type and amount, dispersing solvent type and amount, when present, external environmental conditions, and gel processing, Synerisis can be controlled by properly deciding on the type and level of polymer, fragrance composition, fragrance carrier composition and dispersing solvent, when present.

The composition of the fragrance gels in this invention can be adjusted from soft to stiff products, with no fragrance synerisis.

In one method of preparation, the fragrance is mixed with the carrier solvent at room temperature under well mixing conditions. Once a homogeneous liquid phase formed the required amount of polymer is slowly added under stifling until the mixture starts to thicken. Then, the stirring rate and temperature are slowly increased to keep the mixture homogeneous and flowing. The resulting mixture is poured into molding forms or in carrying containers, such as drums, and cool down to form the gel. When necessary, the solid or the liquid to be dispersed therein is added to the gel in the needed amount during the cooling stage as long as the gel is still in fluid state.

In certain embodiments, the air freshener composition of the present invention has a melting point of at least about 120° F., at least about 130° F., at least 150° F. or at least about 170° F. In alternative embodiments, the melting point can be from about 120° to about 300°, from about 130° to about 260° or from about 150° to about 225°.

In certain embodiments, the air freshener composition of the present invention comprises from about 0.1% to about 90% w/w fragrance, from about 1% to about 50% w/w fragrance, from about 5% to about 40% w/w fragrance or from about 10% to about 30% w/w fragrance.

In a particular embodiment, the air freshener composition may further comprise an active agent. The active agent can include without limitation, a malodor counteracting agents, malodor neutralizing agents, malodor masking agents, dyes, oil-soluble pigments, phosphorescent agents, fluorescent agents, insect repelling compounds, antifungal ingredients, antibacterial actives, antimicrobial actives, UV and light stabilizer ingredients or mixtures thereof.

In certain embodiments, the polymer of the air freshener composition is a normal copolymer, a branched copolymer, a cross-linked copolymer, a radial copolymer or a combination thereof. The copolymer may be, e.g., diblock, triblock, multiblock or a combination thereof. The polymer may also be, e.g., based on thermoplastic rubbers or blends thereof.

The fragrance carrier, when utilized, may be in an amount from about 0.1 to about 99% w/w, from about 1 to about 90% w/w, from about 5 to about 80% w/w or from about 10 to about 75% w/w. The fragrance carrier can be, e.g., a paraffin oil, straight chain hydrocarbon molecule, isoparaffic oil, mineral oil, or combination thereof.

The dispersing agent, when utilized, may be in an amount from about 0.1 to about 80% w/w, from about 1 to about 50% or from about 10% to about 40% w/w. The dispersing agent can be, e.g., an aliphatic compound.

The air freshener composition or device of the present invention may provide a release of the fragrance upon environmental exposure for at least 5 days, at least 7 days, at least 14 days, at least 21 days or at least 30 days.

The fragrance of the composition of the present invention may be released, e.g., by thermo-diffusion, force-induced diffusion or a combination thereof. Thermo-diffusion may be induced by a warmer and the force-induced diffusion may be induced by air circulation (e.g., by the use of a fan).

In certain embodiments, the composition is firm at ambient temperature. In other embodiments, the composition softens at a temperature greater than about 150° F. or greater than about 170° F. In an alternative embodiment, the composition may soften from about 150° F. to about 300° F.

In certain embodiments, the air freshener may further comprise pigments, UV stabilizers, metallic oxides or capsules. The air freshener may also comprise acids, polar liquids, solvents or biologically active molecules.

In certain embodiments, the fragrance carrier comprises a paraffinic oil, an isoparaffinic oil or a combination thereof. In another embodiment the dispersing agent is mineral oil.

In other embodiments, the polymer is an ABC triblock copolymer. The polymer may be a hydrogenated styrene-isoprene-butadiene copolymer. The polymer may also be a styrene elastomer, e.g., selected from styrene-butadiene-styrene elastomer, a styrene-isoprene-styrene elastomer or a combination thereof.

In one embodiment, the polymer of the air-freshener composition comprises an ABC triblock copolymer and a styrene elastomer, e.g., in a ratio of about 1:9 to about 9:1, about 1:5 to about 5:1, about 1:3 to about 3:1 or about 1:1.

In certain embodiments, the carrier and the polymer are in a ratio of about 1:20 to about 20:1, about 1:1 to about 15:1, about 3:1 to about 10:1 or about 6:1 to about 9:1.

In an alternative embodiment, the carrier and the fragrance are in a ratio of about 10:1 to about 1:10, about 1:1 to about 7:1 or about 2:1 to about 5:1.

In a further embodiment, the fragrance and the polymer are in a ratio of about 15:1 to about 1:15, about 10:1 to about 1:1 or about 5:1 to about 1:1.

The following examples are presented to illustrate the invention, and the invention is not to be considered as limited thereto. In the examples, parts are by weight per 100 weight parts of the composition, unless otherwise specified.

Representative examples of fragrance compositions useful in the present invention and sources from which they are available are listed in the table below:

### EXAMPLES

| Example | Fragrance | Source |
|---|---|---|
| 1 | Linen Fresh | AGILEX Fragrances |
| 2 | Green Tea & Aloe | AGILEX Fragrances |
| 3 | Linen Fresh | AGILEX Fragrances |
| 4 | Green Tea & Aloe | AGILEX Fragrances |

In these examples, triblock copolymers of structure ABA, such as Kraton AG-51, ABC such as Septon 4055 or mixture thereof have been used. The fragrance carrier is either a single solvent such as Linpar 1416-V, which is a mixture of saturated linear paraffins, or blends of isoparaffinic oils such as Isopar M and L.

### Example 1

### 25% Fragrance

65% Blend of Isopar M+L (90:10) (isoparaffinic oils)

### 10% Blend of Kraton AG-51+Septon 4055 (50:50)

### Example 2

### 25% Fragrance

65% Linpar 1416-V (hydrogenated paraffins)

### 10% Blend of Kraton AG-51+Septon 4055 (50:50)

### Example 3

### 20% Fragrance

65% Linpar 1416-V (hydrogenated paraffins)
10% Septon 4055 (hydrogenated styrene-isoprene-butadiene copolymer)

### Example 4

### 20% Fragrance

15% Drakeol #5 (light mineral oil NF)
55% Linpar 1416-V (hydrogenated paraffins)
10% Septon 4055 (hydrogenated styrene-isoprene-butadiene copolymer)

While the illustrative embodiments of this invention have been described with particularly, it will be understood that various other modifications will be apparent to and can be readily made by those skilled in the art without departing from the spirit and scope of the invention. Accordingly, it is not intended that the scope of the claims appended hereto to be limited to the examples and descriptions set forth hereinabove but rather that the claims be constructed as encompassing all the features of patentable novelty which reside in the present invention, including all features that would be treated as equivalents thereof by those skilled in the art to which the invention pertains.

1. An air freshener composition comprising a matrix comprising a fragrance dispersed in a thermo-reversible cross-linked polymer, the matrix having a melting point of at least 120° F.

2. The air freshener composition of claim 1, wherein the matrix has a melting point of at least 130° F.

3. The air freshener composition of claim 1, wherein the matrix has a melting point of at least 150° F. or at least 170° F.

4. The air freshener composition of claim 1, comprising from about 0.1% to about 90% w/w fragrance.

5. The air freshener composition of claim 1, comprising from about 1% to about 50% w/w fragrance.

6. The air freshener composition of claim 1, wherein the matrix further comprises an active agent.

7. The air freshener composition of claim 6, wherein the active agent is selected from the group consisting of a malodor counteracting agents, malodor neutralizing agents, malodor masking agents, dyes, oil-soluble pigments, phosphorescent agents, fluorescent agents, insect repelling compounds, antifungal ingredients, antibacterial actives, antimicrobial actives, UV and light stabilizer ingredients and mixtures thereof.

8. The air freshener composition of claim 1, wherein the polymer is a normal copolymer, a branched copolymer, a cross-linked copolymer, a radial copolymer or a combination thereof.

9. The air freshener composition of claim 8, wherein the copolymer is diblock, triblock, multiblock or a combination thereof.

10. The air freshener composition of claim 1, wherein the polymer is based on thermoplastic rubbers or blends thereof.

11. The air freshener composition of claim 10 comprising one or more diblock copolymers, one or more triblock copolymers and a combination thereof.

12. The air freshener composition of claim 1, further comprising a fragrance carrier.

13. The air freshener composition of claim 12, comprising from about 0.1 to about 75% w/w fragrance carrier.

14. The air freshener composition of claim 12 wherein the fragrance carrier is a paraffin oil, straight chain hydrocarbon molecule, isoparaffic oil, mineral oil, or combination thereof.

15. The air freshener composition of claim 1, further comprising a dispersing agent.

16. The air freshener composition of claim 15, comprising from about 0.1 to about 25% dispersing agent.

17. The air freshener composition of claim 16, wherein the dispersing agent is an aliphatic compound.

18. The air freshener composition of claim 1, that provides a release of the fragrance for at least 5 days upon environmental exposure.

19-51. (canceled)

52. An air freshener device comprising an air freshener composition associated with a heat source, wherein the air freshener composition comprises a matrix comprising a fragrance dispersed in a thermo-reversible cross-linked polymer, the matrix having a melting point of at least 120° F.

53-58. (canceled)

59. A method of freshening air comprising heating an air freshener composition such that fragrance is released into the environment, wherein the air freshener composition comprises a matrix comprising a fragrance dispersed in a thermo-reversible cross-linked polymer, the matrix having a melting point of at least 120° F.

60. (canceled)
